(19) United States
(12) Patent Application Publication
(10) Pub. No.: US 2026/0297500 A1
(43) Pub. Date: Oct. 1, 2026

(54) METHOD AND APPARATUS FOR MASS EXTRACTION AND PURIFICATION OF MICROVESICLES FROM BIOLOGICAL SAMPLES

(71) Applicant: Korea University Research and Business Foundation, Seoul (KR)

(72) Inventors: Se-Hyun SHIN, Seoul (KR); Jun-Su PARK, Seoul (KR)

(73) Assignee: Korea University Research and Business Foundation, Seoul (KR)

(21) Appl. No.: 19/168,608

(22) PCT Filed: Feb. 8, 2024
(86) PCT No.: PCT/KR2024/001886

§ 371 Date: Sep. 24, 2025

(30) Foreign Application Priority Data
Mar. 29, 2023 (KR) 10-2023-0041455

(51) Int. Cl. C12M 1/00

(52) U.S. Cl. CPC C12M 47/06; C12M 29/04

(57) ABSTRACT

The present invention relates to a method and apparatus for mass extraction and purification of microvesicles from biological samples, and more particularly, to a method and apparatus for separating and extracting extracellular vesicles from large volumes of culture medium by passing the culture medium through a filter composed of a porous medium whose surface is modified with cationic substances, such that the extracellular vesicles are adsorbed and extracted. According to the present invention, exosomes or extracellular vesicles can be isolated and purified with high purity in an economical and efficient manner. Furthermore, mass extraction and purification of microvesicles can be achieved, making the process suitable for GMP (Good Manufacturing Practice) compliance. As a result, exosomes or extracellular vesicles having high purity and a uniform particle size distribution can be isolated and purified with high yield in a cost-effective and efficient manner.

### TECHNICAL FIELD

The present invention relates to a method and apparatus for mass extraction and purification of microvesicles from biological samples, and more particularly, to a method and apparatus for separating and extracting extracellular vesicles from large volumes of culture medium by passing the culture medium through a filter composed of a porous medium whose surface is modified with a cationic material, thereby allowing the extracellular vesicles to be adsorbed and extracted.

### BACKGROUND ART

Recent studies have reported that cell secretions (secretome) contain a variety of bioactive factors that regulate cellular behavior. In particular, the secretome includes nanosized vesicles, such as exosomes or extracellular vesicles (EVs), which possess intercellular signaling functions. Research on the composition and biological functions of these vesicles has been actively conducted.

Cells release various types of membrane-derived vesicles into the extracellular environment, which are collectively referred to as extracellular vesicles. Extracellular vesicles are also known in the art as cell membrane-derived vesicles, ectosomes, shedding vesicles, microparticles, or exosomes, and in some cases the terms are distinguished from one another. In the present invention, the term microvesicles is defined to encompass both extracellular vesicles and exosomes.

Exosomes are vesicles having a lipid bilayer membrane identical to the cell membrane, with sizes ranging from several tens to several hundreds of nanometers. Inside the exosomes are cargos referred to as exosome cargos, which include proteins, mRNAs, and miRNAs. The exosome cargos contain a wide variety of signaling factors that are known to be cell type-specific and regulated depending on the microenvironment of the secreting cells.

Exosomes act as mediators of intercellular communication. The cellular signals delivered via exosomes regulate cellular behaviors of recipient cells, including activation, growth, migration, differentiation, dedifferentiation, apoptosis, and necrosis. Moreover, depending on the origin and physiological state of the secreting cells, exosomes contain specific genetic materials and bioactive factors. For example, exosomes derived from proliferating stem cells regulate cellular migration, proliferation, and differentiation, and reflect the regenerative properties of stem cells (see Nature Reviews Immunology, 2002, Vol. 2, pp. 569-579).

Conventional techniques for isolating exosomes or extracellular vesicles include ultracentrifugation, density gradient centrifugation, ultrafiltration, size exclusion chromatography, ion exchange chromatography, immunoaffinity capture, microfluidics-based isolation, exosome precipitation, commercial total exosome isolation kits, polymer-based precipitation, and tangential flow filtration (TFF).

Ultracentrifugation has been the most widely used method for the isolation of exosomes or extracellular vesicles. However, it suffers from low yield, long processing times, labor-intensive procedures, and the requirement for expensive equipment. Moreover, ultracentrifugation may cause physical damage to exosomes or extracellular vesicles during the separation process, thereby adversely affecting subsequent analysis or applications.

Ultrafiltration, often combined with ultracentrifugation, may improve purity but reduces yield due to non-specific adhesion of vesicles to the filter membranes. Immunoaffinity capture, while providing high specificity by binding exosomes or extracellular vesicles with antibodies, requires antibody production and subsequent removal steps, making the process expensive and unsuitable for scale-up.

More recently, commercial exosome isolation kits, such as precipitation-based methods or polymer-based isolation kits, have become available. Although convenient, these methods rely on costly reagents and are unsuitable for large-scale isolation and purification of exosomes or extracellular vesicles.

Tangential flow filtration (TFF) allows continuous processing of large sample volumes by passing fluids tangentially along the membrane surface. While suitable for large-scale operation, the recovery yield is known to be significantly lower compared with precipitation-based methods.

The major challenge in existing methods is the co-isolation of impurities, such as proteins, which results in low purity and yield of the isolated exosomes or extracellular vesicles. In addition, conventional separation and purification methods are time-consuming, cumbersome, and expensive. Methods developed to increase purity often make scale-up difficult and are incompatible with Good Manufacturing Practice (GMP) requirements.

Accordingly, there is a continuous demand in the field for technologies that can isolate and purify exosomes or extracellular vesicles with high purity in an economical and efficient manner, while being suitable for scale-up and compliant with GMP. In other words, there is a need for novel technologies that enable the large-scale, cost-effective, and efficient isolation and purification of exosomes or extracellular vesicles with high purity and uniform particle size distribution.

### DETAILED DESCRIPTION

### Problems to be Solved

In order to overcome the aforementioned problems, an object of the present invention is to provide a method and apparatus for mass extraction and purification of microvesicles from biological samples. The invention utilizes the negatively charged properties of extracellular vesicles and exosomes generated in large-volume culture medium, by attaching cationic substances to capture surfaces such as microparticles or membranes, thereby enabling effective adsorption of the microvesicles onto the surfaces.

Another object of the present invention is to provide a method and apparatus for mass extraction and purification of microvesicles from biological samples, wherein the capture efficiency of the microvesicles is significantly improved by configuring the filtration unit with a porous medium having microstructures, thereby increasing the effective surface area available for adsorption of the microvesicles.

A further object of the present invention is to provide a method and apparatus for mass extraction and purification of microvesicles from biological samples, wherein the porous medium is implemented in the form of a filter, thereby enabling continuous processing of large volumes of culture medium and improving the efficiency of extraction and purification of the microvesicles.

### Means to Solve Problems

According to one aspect of the present invention conceived to solve the above-described problems, there is provided a method for extracting microvesicles from a microvesicle solution, wherein the microvesicle solution is passed through a microfilter structure (6) so that the microvesicles are electrostatically bound to the microfilter structure (6).

The method further comprises introducing an elution solution into the microfilter structure (6) to which the microvesicles are bound, thereby separating the microvesicles electrostatically bound to the microfilter structure (6).

The microfilter structure (6) may be formed of a microporous structure selected from the group consisting of a membrane structure, a mesh structure, and a stacked structure of spherical beads.

The membrane structure or mesh structure of the microfilter structure (6) may be configured as a multilayer structure comprising two or more microporous membranes laminated together.

The surface of the microporous structure is modified with a polycationic substance.

The polycationic substance may be one or more selected from the group consisting of polylysine, protamine, polyarginine, polyhistidine, cationic dextran, cationic dendrimers, cationic polysaccharides, polyamidoamines, polyethyleneimines, polyquaterniums, poly(2-dimethylaminoethyl methacrylate) (PDMAEMA), poly(2-dimethylaminomethyl styrene) (PDMAMS), poly(1-vinylpyrrolidone) (p1-VP), poly(diethylaminoethyl acrylate) (pDEAEA), poly(dimethylaminoethyl acrylate) (pDMAEA), poly(diethylaminoethyl methacrylate) (pDEAMA), lipopolyamines, quaternary ammoniums, guanidine, imidazole, polyaniline, polypyrrole, and chitosan.

The step of separating the microvesicles by introducing the elution solution comprises: introducing a first reagent into the microfilter structure (6) and washing the microfilter structure under a first condition; and introducing a second reagent into the microfilter structure (6) and washing the microfilter structure under a second condition. The first reagent is a substance including one or more selected from Cl⁻, CH₃COO⁻, SO₄²⁻, HCO₃⁻, SiO₃²⁻, and OH⁻, and the first condition is set to be performed at least once within a pH range of 5.5 to 6.5 or 7.5 to 9.5.

The second reagent is a substance including one or more selected from Cl⁻, CH₃COO⁻, SO₄²⁻, HCO₃⁻, SiO₃²⁻, and OH⁻ at a concentration of 0.01 M to 3 M, and the second condition is set to be performed at least once for 1 to 60 minutes while mixing at 500 rpm to 2000 rpm.

The microporous structure may be made of one or more selected from polyacrylates, polyacrylamides, polymethacrylates, polyethylene glycols, polystyrene-divinylbenzenes, polystyrenes, hydrogels, agarose, ceramics, silica, latex, metal particles, glass particles, and magnetic particles.

The membrane structure and the mesh structure may be made of one or more selected from nylon, polyethylene, polypropylene, polyethylene terephthalate (PET), polylactic acid (PLA), polyethersulfone (PES), cellulose acetate (CA), polyvinylidene fluoride (PVDF), and polycarbonate (PC).

According to another embodiment of the present invention, there is provided a method for extracting microvesicles from a microvesicle solution, comprising: a first step of preparing a microvesicle solution containing microvesicles to be extracted and purified;
a second step of primarily filtering the microvesicle solution;
a third step of secondarily filtering the primarily filtered microvesicle solution; and
a fourth step of introducing an elution solution into the secondarily filtered microvesicle solution to separate the microvesicles.

In the second step, the microvesicle solution is passed through a pre-filter (4) to remove one or more impurities, cell debris, or proteins included in the microvesicle solution. In the third step, the microvesicle solution is passed through the microfilter structure (6) so that the microvesicles are electrostatically bound to the microfilter structure (6). In the fourth step, an elution solution is introduced into the microfilter structure (6) to which the microvesicles are bound, thereby separating the microvesicles bound to the microfilter structure (6).

According to yet another embodiment of the present invention, there is provided an apparatus for extracting microvesicles from a microvesicle solution, comprising:
a first supply container (1) for storing and supplying a microvesicle solution containing microvesicles to be extracted and purified;
a pre-filter (4) for primarily filtering the microvesicle solution introduced from the first supply container (1) to remove one or more impurities, cell debris, or proteins;
a microfilter structure (6) for secondarily filtering the microvesicle solution introduced from the pre-filter (4) in the primarily filtered state, so that the microvesicles are electrostatically bound thereto;
a second supply container (11) for storing and supplying an elution solution for separating the microvesicles bound to the microfilter structure (6); and
a second storage container (12) for storing the elution solution containing the microvesicles separated from the microfilter structure (6).

### Advantageous Effects

According to the present invention, exosomes or extracellular vesicles can be isolated and purified with high purity in an economical and efficient manner. Furthermore, large-scale isolation and purification of microvesicles can be achieved, thereby making the process suitable for GMP (Good Manufacturing Practice) compliance. As a result, exosomes or extracellular vesicles having high purity and a uniform particle size distribution can be isolated and purified with high yield, economically and efficiently.

### BRIEF DESCRIPTION OF THE DRAWINGS

FIG. 1 is a conceptual diagram showing the structure of a microvesicle extraction apparatus according to an embodiment of the present invention.

FIG. 2 is a flowchart illustrating the process of a microvesicle extraction method according to the present invention.

FIG. 3 is a conceptual diagram illustrating the filtration process of microvesicles.

FIG. 4 is a conceptual diagram illustrating the separation process of microvesicles.

FIG. 5 is a cross-sectional view showing the structure of a microfilter structure.

FIG. 6 is a cross-sectional view showing a state in which microvesicles are adsorbed onto the microfilter structure.

FIG. 7 is a cross-sectional view showing a state in which microvesicles are adsorbed onto a mesh filter unit.

FIG. 8 is a graph showing extraction concentration results according to the number of stacked mesh-structured microfilter structures.

FIG. 9 is a graph showing nucleic acid expression results according to the number of stacked mesh-structured microfilter structures.

**Descriptions of Reference Numerals**

| | |
|---|---|
| 1: first supply container | 2: transfer pipe |
| 3: first pump | 4: pre-filter |
| 5: first valve | 6: microfilter structure |
| 7: second pump | 8: second valve |
| 9: first storage container | 10: third pump |
| 11: second supply container | 12: second storage container |

### MODES OF INVENTION

An exemplary embodiment of the present invention will be described with reference to the accompanying drawings, and an object and the configuration, and the features of the present invention will be understood well through the detailed description.

The exemplary embodiment described above is only to describe exemplary embodiment of the present invention and is not limited to the exemplary embodiment, and various modifications and variations are possible by those skilled in the art within the spirit and claims of the present invention, and it will be said that the modifications and variations fall within the scope of the technical rights of the present invention.

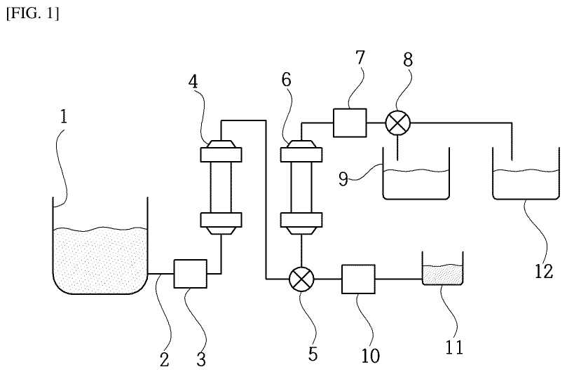

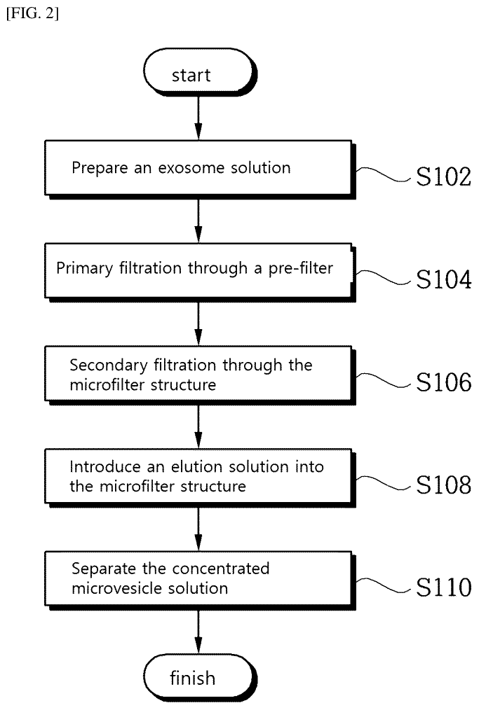

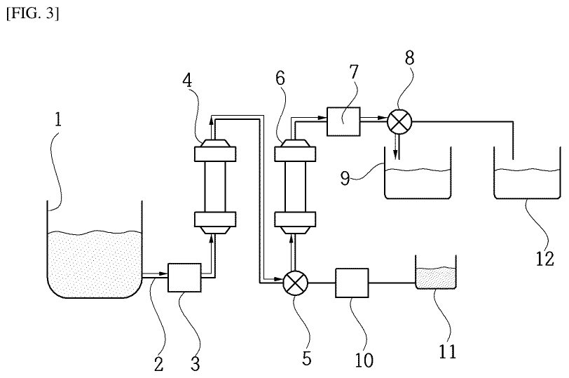

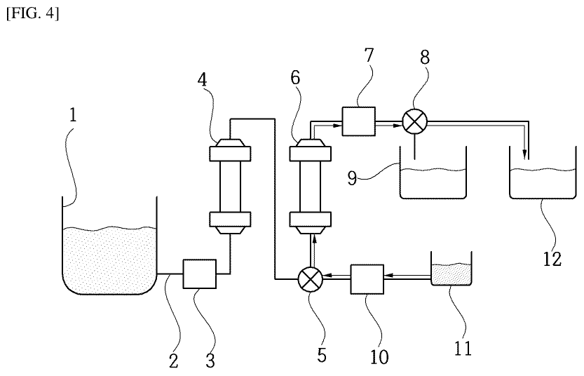

FIG. 1 is a conceptual diagram showing the structure of a microvesicle extraction apparatus according to an embodiment of the present invention. FIG. 2 is a flowchart illustrating the process of a microvesicle extraction method of the present invention. FIG. 3 is a conceptual diagram illustrating the filtration process of microvesicles, and FIG. 4 is a conceptual diagram illustrating the separation process of microvesicles.

The present invention relates to a technology for mass extraction and purification of extracellular vesicles or exosomes from biological samples containing cells, viruses, or the like. In particular, the invention is characterized by applying a two-step filtration process and a microvesicle separation process while continuously introducing a large volume of culture medium.

For this purpose, the “apparatus for mass extraction and purification of microvesicles from biological samples” (hereinafter referred to as the “extraction apparatus”), as illustrated in FIG. 1, is applied to the extraction and purification process. As shown in FIG. 1, the extraction apparatus of the present invention may comprise: a means for supplying a large volume of microvesicle solution (a solution in which extracellular vesicles or exosomes are cultured), a means for filtering microvesicles from the microvesicle solution, and a means for separating microvesicles from the filter after filtration.

A first supply container (1) stores the microvesicle solution and continuously supplies the solution through a transfer pipe (2). Each component included in the extraction apparatus of the present invention is connected through the transfer pipe (2), and unless otherwise specified, it is to be understood that such connection is made by the same structure of transfer pipe (2).

A first pump (3) conveys a large volume of the microvesicle solution stored in the first supply container (1) to a subsequent filter by generating positive or negative pressure.

The inlet of a pre-filter (4), which is connected to the first pump (3) through the transfer pipe (2), receives the microvesicle solution. The pre-filter (4) primarily removes impurities, cell debris, or proteins contained in the microvesicle solution. As the pre-filter (4), membranes, meshes, microparticle layers, or porous materials having pores smaller than the average size of the target substances to be filtered may be applied. Substances such as impurities having a size larger than the pores formed in the pre-filter (4) cannot pass through and are removed.

The outlet of the pre-filter (4) is connected via the transfer pipe (2) to a first valve (5). Preferably, the first valve (5) has a three-way valve structure. In general, the first valve (5) comprises two inlets and one outlet. The transfer pipe (2) connected to the outlet of the pre-filter (4) is connected to the first of the two inlets. By the opening and closing operation of the first valve (5), the two inlets may be selectively connected to the single outlet.

The outlet of the first valve (5) is connected to the inlet of a microfilter structure (6). Therefore, the microvesicle solution that has passed through the pre-filter (4), with impurities removed, flows through the first valve (5) and is introduced into the microfilter structure (6).

The microfilter structure (6) is formed of a microporous structure having a plurality of pores. As the microvesicle solution passes through the microporous structure, the microvesicles are retained within the microporous structure, thereby achieving filtration.

The surface of the microporous structure is manufactured in a form in which a substance having a specific charge is attached, so that the microvesicles are bound to the surface of the material in the microfilter by electrostatic interaction. Typically, extracellular vesicles or exosomes contained in the microvesicle solution possess a negative charge. Therefore, it is preferable that the surface of the material forming the microporous structure inside the microfilter structure (6) be modified with polycationic substances having positive charge characteristics.

The polycationic substance used for surface modification of the microfilter structure (6) may be one or more selected from the group consisting of polylysine, protamine, polyarginine, polyhistidine, cationic dextran, cationic dendrimers, cationic polysaccharides, polyamidoamines, polyethyleneimines, polyquaterniums, poly(2-dimethylaminoethyl methacrylate) (PDMAEMA), poly(2-dimethylaminomethyl styrene) (PDMAMS), poly(l-vinylpyrrolidone) (p1-VP), poly(diethylaminoethyl acrylate) (pDEAEA), poly(dimethylaminoethyl acrylate) (pDMAEA), poly(diethylaminoethyl methacrylate) (pDEAMA), lipopolyamines, quaternary ammoniums, guanidine, imidazole, polyaniline, polypyrrole, and chitosan.

The microfilter structure (6) may be formed in a structure selected from a microparticle structure including a stacked structure of spherical beads, a membrane structure, or a mesh structure.

In the case of the microparticle structure, voids are formed inside the stacked structure so that, as the microvesicle solution passes through, the microvesicles contained therein are electrostatically bound to the surfaces of the microparticles. Since the cationic substances attached to the surfaces of the microparticles attract and bind the negatively charged microvesicles through electrostatic interaction, it is not necessary for the void size between the microparticles to be smaller than the diameter of the microvesicles. Rather, it is preferable that the voids be large enough to allow smooth passage of the microvesicle solution.

Although the particle size of the microparticles and the voids may vary depending on the material, it is preferable that the ratio of the diameter of the microvesicles to that of the spherical microparticles be in the range of 1:5 to 1:10. When microparticles of such sizes are used, the voids formed between the microparticles may have a size approximately two to five times greater than the diameter of the microvesicles.

The microparticles used in the present invention may be made of one or more selected from polyacrylates, polyacrylamides, polymethacrylates, polyethylene glycols, polystyrene-divinylbenzenes, polystyrenes, hydrogels, agarose, ceramics, silica, latex, metal particles, glass particles, and magnetic particles.

The microfilter structure (6) may also be formed in a membrane structure or a mesh structure, in which case a material capable of adsorbing polycationic substances is used. Examples of the materials for the membrane or mesh include nylon, polyethylene, polypropylene, polyethylene terephthalate (PET), polylactic acid (PLA), polyethersulfone (PES), cellulose acetate (CA), polyvinylidene fluoride (PVDF), and polycarbonate (PC). Similar to the microparticle structure, the membrane or mesh structure is fabricated in a state in which polycationic substances are adsorbed onto the surface.

The membrane structure or mesh structure may be a single layer, or, in some cases, may be configured as a multilayer to enhance filtration efficiency. When configured as a multilayer, it is preferable that the structure be formed by laminating ten or more single layers.

The transfer pipe (2) connected to the outlet of the microfilter structure (6) is connected to a second pump (7). The second pump (7) causes the microvesicle solution or the elution solution to pass through the microfilter structure (6) by generating positive or negative pressure.

The second pump (7) is connected to a second valve (8). Preferably, the second valve (8) has a three-way valve structure similar to that of the first valve (5), although the number of inlets and outlets may be configured differently. Typically, the second valve (8) comprises one inlet and two outlets, and the transfer pipe (2) connected to the outlet of the microfilter structure (6) is connected to the single inlet. By the switching operation of the second valve (8), the two outlets can be selectively connected to the single inlet.

By the switching operation of the second valve (8), one of the two outlets is connected to a first storage container (9). The microvesicle solution that has passed through the microfilter structure (6) is introduced into the first storage container (9) via the second valve (8) in a state where the microvesicles have been removed. Since most of the extracellular vesicles and exosomes contained in the microvesicle solution are adsorbed to the microfilter structure (6), only a trace amount of extracellular vesicles and exosomes remain in the solution stored in the first storage container (9).

Meanwhile, a third pump (10) is connected to the second inlet of the two inlets of the first valve (5). The third pump (10) is further connected to the outlet of a second supply container (11). Accordingly, the elution solution stored in the second supply container (11) is introduced into the microfilter structure (6) through the first valve (5) under the pressure generated by the third pump (10).

The elution solution introduced into the microfilter structure (6) dissolves the microvesicles adsorbed onto the microparticles, membranes, or meshes therein. The elution solution containing the dissolved microvesicles then passes through the second valve (8) and is introduced into a second storage container (12). For this purpose, the second valve (8) is switched so that the flow path is formed through the second of the two outlets. As a result, the elution solution is stored in the second storage container (12).

The elution process using the elution solution may be carried out as a single step. However, in order to improve elution efficiency, it may also be performed as a multistep process. In the present invention, the elution efficiency can be enhanced by sequentially introducing two different types of elution solutions.

In a first step, the microfilter structure (6) is subjected to a primary washing under a first condition using a first reagent. The first reagent may include one or more substances selected from Cl⁻, CH₃COO⁻, SO₄²⁻, HCO₃⁻, SiO³²⁻, and OH⁻. The first condition may be set such that the washing is performed at least once within a pH range of 5.5 to 6.5 or 7.5 to 9.5.

In a second step, the microfilter structure (6) is subjected to a secondary washing under a second condition using a second reagent. The second reagent may include one or more substances selected from Cl⁻, CH₃COO⁻, SO₄²⁻, HCO₃⁻, SiO³²⁻, and OH⁻, and is preferably used at a concentration in the range of 0.01 M to 3 M. The second condition may be set such that the washing is performed at least once for a period of 1 to 60 minutes while mixing at 500 rpm to 2000 rpm.

The extraction method and its sequence using the above-described extraction apparatus are the same as illustrated in FIG. 2.

First, a microvesicle solution containing the microvesicles to be extracted and purified is prepared (S102).

Next, while operating the first pump (3) or the second pump (7), the microvesicle solution stored in the first supply container (1) is filtered through the pre-filter (4) and the microfilter structure (6), and the discharged solution is stored in the first storage container (9) (S104, S106). In this state, the first valve (5) is controlled so that a flow path is formed between the pre-filter (4) and the microfilter structure (6), and the second valve (8) is controlled so that a flow path is formed between the second pump (7) and the first storage container (9).

When the filtration through the microfilter structure (6) is completed, the elution solution stored in the second supply container (11) is introduced into the microfilter structure (6) (S108). In this state, the first valve (5) is switched so that a flow path is formed between the third pump (10) and the microfilter structure (6), and the second valve (8) is switched so that a flow path is formed between the second pump (7) and the second storage container (12).

As described above, the elution process may be carried out in two stages: a step of introducing the first reagent under the first condition, and a step of introducing the second reagent under the second condition.

Finally, by subjecting the separated solution stored in the second storage container (12), in which the microvesicles have been separated, to a separate process, the extraction and purification of the microvesicles is completed.

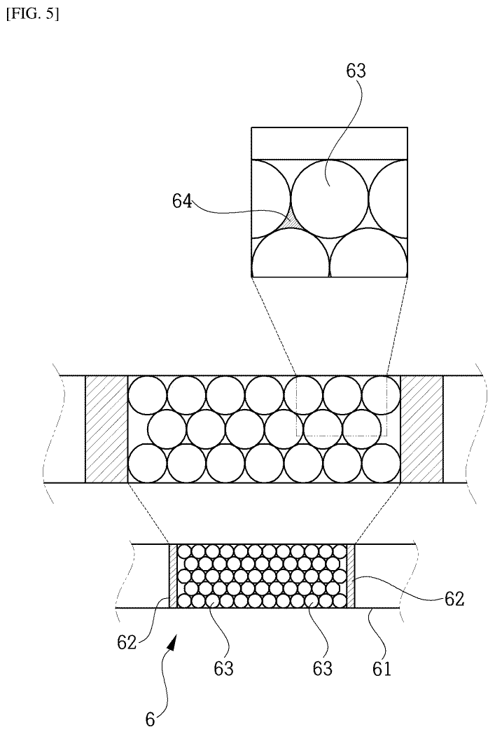

Meanwhile, FIG. 5 is a cross-sectional view illustrating the structure of the microfilter structure, and FIG. 6 is a cross-sectional view illustrating a state in which microvesicles are adsorbed onto the microfilter structure.

As described above, when the filter membrane (61) is formed of a microparticle structure (63), the voids (64) between the microparticles (63) are formed to be relatively larger than the size of the microvesicles (EVs). The microparticles (63) are stacked between the filter membrane (61) and the sidewall (62). The filter membrane (61) may be formed as a multilayer.

The microvesicles (EVs) are electrostatically bound by cationic substances adsorbed on the surfaces of the microparticles (63). Since the size of the microvesicles is relatively smaller than that of the voids (64), the microvesicle solution can smoothly pass through even in a state where the microvesicles are sufficiently adsorbed.

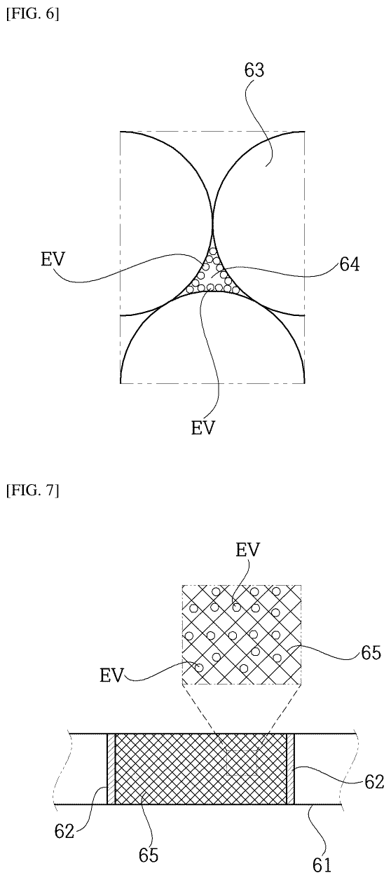

Meanwhile, FIG. 7 is a cross-sectional view illustrating a state in which microvesicles are adsorbed onto a mesh filter unit. Even in the case of a membrane structure or a mesh structure, the voids formed by the lattice-shaped yarns (65) are still considerably larger than the size of the microvesicles. Therefore, the microvesicle solution can smoothly flow through even when the microvesicles are sufficiently adsorbed onto the yarns (65).

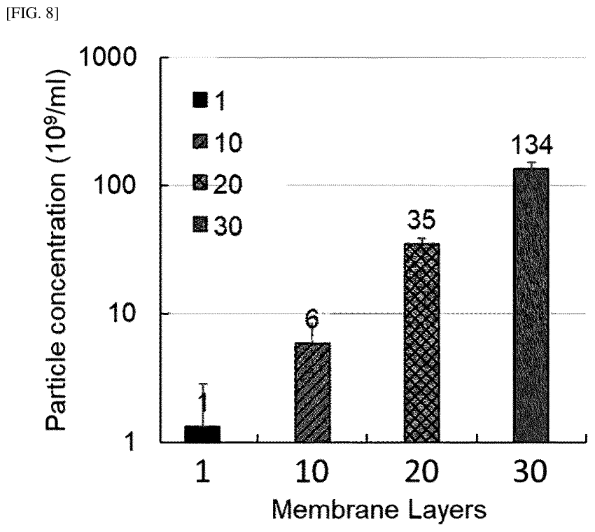

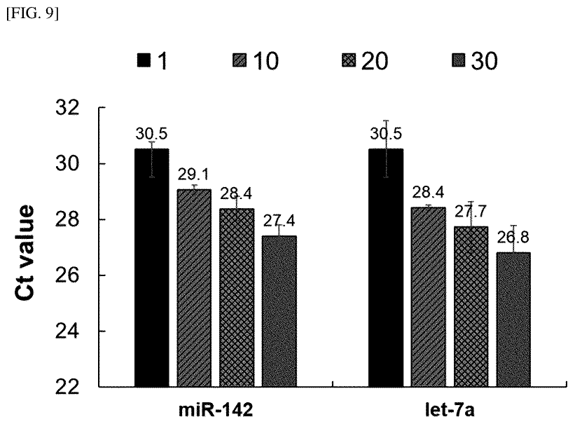

FIG. 8 is a graph showing the extraction concentration results according to the number of stacked layers of the mesh-structured microfilter structure, and FIG. 9 is a graph showing nucleic acid expression results according to the number of stacked layers of the mesh-structured microfilter structure.

Specifically, FIG. 8 illustrates the particle concentration results, indicating the number of extracellular vesicles recovered depending on the number of stacked mesh layers. FIG. 9 illustrates the nucleic acid amplification results for two specific miRNAs present inside the recovered extracellular vesicles, depending on the number of stacked mesh layers. In this experiment, the data were obtained from the analysis of 1 mL of plasma.

As illustrated in FIG. 8, the graph shows that when the microporous membrane structure composed of a mesh is configured as a single layer (1 sheet) and compared with multilayers (in particular, 10, 20, and 30 sheets), the recovery rate of extracellular vesicles increases exponentially. In other words, as the number of stacked microporous membrane layers increases, the number of microvesicles extracted per unit mass also increases.

As illustrated in FIG. 9, the graph shows the change in ΔCt values when the microporous membrane structure composed of a mesh is configured as a single layer (1 sheet) and compared with multilayers (10, 20, and 30 sheets). Specifically, as the number of stacked microporous membrane layers increases, the nucleic acid content of the recovered microvesicles also increases, thereby reducing the number of PCR cycles required to reach a given amplification threshold.

As can be seen from FIGS. 8 and 9, when the microporous membrane is configured as a multilayer rather than a single layer, the recovery rate of microvesicles and the like is significantly improved as the number of stacked layers increases. Extraction experiments using plasma indicate that forming approximately 30 to 50 layers of microporous membranes is considered to be the most efficient.

Furthermore, the performance improvement achieved by multilayer formation of the microporous membrane structure can also be applied to membrane-type microporous structures. In the case of a membrane structure as well, configuring the structure as a multilayer rather than a single layer enhances the extraction and recovery efficiency of the microvesicles.

By using the extraction apparatus and extraction method having such a structure, it is expected that a device capable of processing more than 300 liters of solution, at a throughput of 100 liters or more per hour, can be developed.

What is claimed is:

1. A method for extracting microvesicles from a microvesicle solution,
comprising passing the microvesicle solution through a microfilter structure (6) such that the microvesicles are electrostatically bound to the microfilter structure (6).

2. The method of claim 1,
wherein an elution solution is introduced into the microfilter structure (6) to which the microvesicles are bound, thereby separating the microvesicles bound to the microfilter structure (6).

3. The method of claim 1,
wherein the microfilter structure (6) is formed of a microporous structure selected from the group consisting of a membrane structure, a mesh structure, and a stacked structure of spherical beads.

4. The method of claim 3,
wherein the membrane structure or the mesh structure of the microfilter structure (6) comprises a multilayer formed by laminating two or more microporous membranes.

5. The method of claim 3,
wherein the surface of the microporous structure is modified with a polycationic substance.

6. The method of claim 5,
wherein the polycationic substance is one or more selected from the group consisting of polylysine, protamine, polyarginine, polyhistidine, cationic dextran, cationic dendrimers, cationic polysaccharides, polyamidoamines, polyethyleneimines, polyquaterniums, poly(2-dimethylaminoethyl methacrylate) (PDMAEMA), poly(2-dimethylaminomethyl styrene) (PDMAMS), poly(l-vinylpyrrolidone) (p1-VP), poly(diethylaminoethyl acrylate) (pDEAEA), poly(dimethylaminoethyl acrylate) (pDMAEA), poly(diethylaminoethyl methacrylate) (pDEAMA), lipopolyamines, quaternary ammoniums, guanidine, imidazole, polyaniline, polypyrrole, and chitosan.

7. The method of claim 2,
wherein the step of separating the microvesicles by introducing the elution solution comprises:
introducing a first reagent into the microfilter structure (6) and washing the microfilter structure under a first condition; and
introducing a second reagent into the microfilter structure (6) and washing the microfilter structure under a second condition.

8. The method of claim 7,
wherein the first reagent comprises one or more substances selected from Cl⁻, CH₃COO⁻, SO₄²⁻, HCO₃⁻, SiO³²⁻, and OH⁻, and
the first condition is set to be performed at least once within a pH range of 5.5 to 6.5 or 7.5 to 9.5.

9. The method of claim 7,
wherein the second reagent comprises one or more substances selected from Cl⁻, CH₃COO⁻, SO₄²⁻, HCO₃⁻, SiO³²⁻, and OH⁻ at a concentration of 0.01 M to 3 M, and
the second condition is set to be performed at least once for a period of 1 to 60 minutes while mixing at 500 rpm to 2000 rpm.

10. The method of claim 5,
wherein the microporous structure comprises one or more materials selected from polyacrylates, polyacrylamides, polymethacrylates, polyethylene glycols, polystyrene-divinylbenzenes, polystyrenes, hydrogels, agarose, ceramics, silica, latex, metal particles, glass particles, and magnetic particles.

11. The method of claim 3,
wherein the membrane structure and the mesh structure comprise one or more materials selected from nylon, polyethylene, polypropylene, polyethylene terephthalate (PET), polylactic acid (PLA), polyethersulfone (PES), cellulose acetate (CA), polyvinylidene fluoride (PVDF), and polycarbonate (PC).

12. A method for extracting microvesicles from a microvesicle solution,
comprising:
preparing a microvesicle solution containing microvesicles to be extracted and purified;
primarily filtering the microvesicle solution;
secondarily filtering the primarily filtered microvesicle solution; and
introducing an elution solution into the secondarily filtered microvesicle solution to separate the microvesicles.

13. The method of claim 12,
wherein the primary filtering step comprises passing the microvesicle solution through a pre-filter (4) to remove one or more impurities, cell debris, or proteins contained in the microvesicle solution.

14. The method of claim 12,
wherein the secondary filtering step comprises passing the microvesicle solution through the microfilter structure (6) such that the microvesicles are electrostatically bound to the microfilter structure (6).

15. The method of claim 14,
wherein the elution step comprises introducing an elution solution into the microfilter structure (6) to which the microvesicles are bound, thereby separating the microvesicles bound to the microfilter structure (6).

16. An apparatus for extracting microvesicles from a microvesicle solution,
comprising:
a first supply container (1) for storing and supplying a microvesicle solution containing microvesicles to be extracted and purified;
a pre-filter (4) for primarily filtering the microvesicle solution introduced from the first supply container (1) to remove one or more impurities, cell debris, or proteins;
a microfilter structure (6) for secondarily filtering the microvesicle solution introduced from the pre-filter (4) in a primarily filtered state, such that the microvesicles are electrostatically bound thereto;
a second supply container (11) for storing and supplying an elution solution for separating the microvesicles bound to the microfilter structure (6); and
a second storage container (12) for storing the elution solution containing the microvesicles separated from the microfilter structure (6).
